(19) United States
(12) Patent Application Publication
(10) Pub. No.: US 2005/0171476 A1
(43) Pub. Date: Aug. 4, 2005

(54) Medication injecting apparatus with fluid container piston-engaging drive member having internal hollow for accommodating drive member shifting mechanism

(76) Inventors: Jared Alden Judson, Topsfield, MA (US); Kenneth Alan Ritsher, Chicago, IL (US)

Correspondence Address: ELI LILLY AND COMPANY;PATENT DIVISION, P.O. BOX 6288, INDIANAPOLIS, IN, 46206-6288, US

(21) Appl. No.: 10/515,167

(22) PCT Filed: May 21, 2003
(86) PCT No.: PCT/US03/14538

(30) Foreign Application Priority Data
May 24, 2002 (US) 60383558

(51) Int. Cl. A61M 37/00

(52) U.S. Cl. 604/131

(57) ABSTRACT

A medication injecting apparatus having a motor-driven drive member that when advanced inserts into a fluid container for forcing fluid therefrom. The drive member includes an internal hollow in which fits at least a portion of the motorized driver assembly when the drive member is retracted to allow a compact apparatus to be provided.

### BACKGROUND OF THE INVENTION

The present invention pertains to medication injecting devices, and, in particular, to a portable medication injecting device such as an injection pen.

Patients suffering from a number of different diseases frequently must inject themselves with medication. To allow a person to conveniently and accurately self-administer medicine, a variety of devices broadly known as injector pens or injection pens have been developed. Generally, these pens are equipped with a cartridge including a piston and containing a multi-dose quantity of liquid medication. A drive member of the pen is movable forward to advance the piston in the cartridge in such a manner to dispense the contained medication from an outlet at the opposite cartridge end, typically through a needle that penetrates a stopper at that opposite end.

A variety of electromechanical injection pens have been developed which utilize an electric motor to set and/or administer a dose of medication. For example, one such electromechanical injection pen utilizes a motor to arrange the pen to deliver a particular dose, which dose can be subsequently administered by a manually powered advancement of the drive member. One possible problem with electromechanical injection pens is that the inclusion of a motor may unacceptably increase the overall size of the pen. Many potential users may be put off by larger sized pens, for reasons including the pens being more bulky and unwieldy, which may make inconvenient their use or transport.

Thus, it would be desirable to provide an apparatus that can overcome one or more of these and other shortcomings of the prior art.

### BRIEF SUMMARY OF THE INVENTION

In one form thereof, the present invention encompasses a medication injecting apparatus including a housing, a drive member movable in a distal direction relative to the housing from a retracted position to an extended position, a fluid container defining a medicine-filled reservoir with a movable piston at one end and an outlet at the other end, which piston is engagable by the drive member to be shifted distally toward the outlet when the drive member is moved distally, an electronic circuit, and a motorized driver assembly controlled by the electronic circuit and coupled with the drive member for selectively shifting the drive member distally. The drive member includes a tubular body that inserts within the fluid container when advancing from the retracted position to the extended position, and at least a portion of the motorized driver assembly fits within an internal hollow of the tubular body when the drive member is disposed in the retracted position.

In another form thereof, the present invention encompasses a medication injecting apparatus including a housing, a drive member movable in a distal direction relative to the housing from a retracted position to an extended position, a replaceable cartridge defining a medicine-filled reservoir with a movable piston at one end and an outlet at the other end, the piston being engagable by the drive member to be shifted distally toward the outlet when the drive member is moved distally, an electronic circuit, and a motorized driver assembly including an electric motor, which motorized driver assembly is controlled by the electronic circuit and coupled with the drive member for selectively shifting the drive member distally. Along its length extending in the distal direction, the motorized driver assembly is aligned generally coaxially with the cartridge, and along at least a substantial portion of its length, the motorized driver assembly has a transverse cross-sectional shape dimensioned so as to be fittable within the space occupied by the cartridge.

One advantage of the present invention is that a medication injecting apparatus can be provided which utilizes a motorized driver assembly to inject a dose of medication.

Another advantage of the present invention is that a medication injecting apparatus can be provided which has a motorized driver assembly of compact size that can contribute to a small overall size of the apparatus.

Still another advantage of the present is that a medication injecting apparatus can be provided in which part or all of a motorized driver assembly used to shift a cartridge piston-engaging drive member can be housed within an internal hollow of the drive member when the drive member is fully retracted.

Still another advantage of the present is that a medication injecting apparatus can be provided in which a microcontroller is included, which microcontroller allows for warnings, such as an abnormally high or low dose, user feedback via sounds or lights and clock or alarm features.

### BRIEF DESCRIPTION OF THE DRAWINGS

The above-mentioned and other advantages and objects of this invention, and the manner of attaining them, will become more apparent, and the invention itself will be better understood by reference to the following description of embodiments of the invention taking in conjunction with the accompanying drawings, wherein:

FIG. 1 is a diagrammatic front view of a first embodiment of an electromechanical medication injecting apparatus with hollow drive member of the present invention;

FIG. 2 is a schematic showing components of the electromechanical medication injecting apparatus of FIG. 1;

FIG. 3 is a diagrammatic view in partial cross-section of select components of the apparatus of FIG. 2, and wherein the apparatus is shown with the drive member extended farther distally than in FIG. 2; and

FIG. 4 is a schematic showing components of an alternate embodiment of the present invention.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

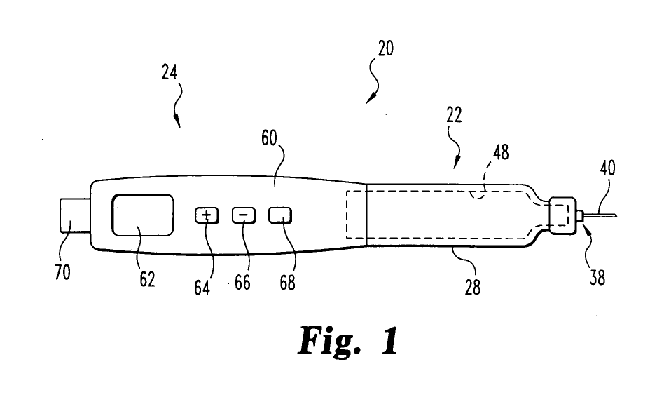

Referring now to FIG. 1, there is shown a first embodiment of a medication injecting apparatus of the present invention. The apparatus, generally designated 20, is shown in a form commonly known as a reusable injection pen, although other forms of portable medication injecting apparatuses, including injection pens having more box-like shapes, and less writing pen-like shapes, are within the scope of the invention.

Injection pen 20 includes a distal portion 22 that contains the medicinal fluid to be delivered upon pen operation, and a proximal portion 24 that contains the mechanisms used to force the contained medicine from the distal portion. In the shown embodiment, distal portion 22 includes a retainer 28 and a replaceable cartridge 48 held therein. Cartridge retainer 28 may be made of a transparent plastic, or provided with one or more viewing windows, to allow the cartridge contents to be visible. Cartridge retainer 28 is removably mounted, such as with a threaded connection, to pen proximal portion 24.

A replaceable pen-needle assembly 38 of known design is shown connected, such as with a threaded connection, to the distal end of retainer 28. Pen-needle assembly 38 includes a double-ended needle cannula or injection needle 40.

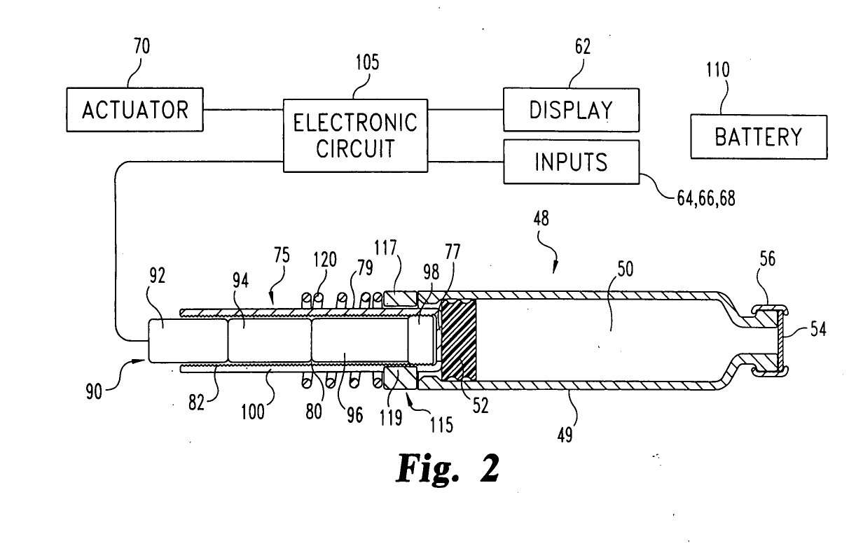

With additional reference to FIG. 2, cartridge 48 is a standard 3 ml cartridge and has a glass or plastic housing 49. Housing 49 defines a medicine-filled reservoir 50 that is closed at its proximal end by a disc-shaped piston 52 that is axially slidably and sealably engaged with the cartridge housing interior wall to hold the fluid medication within reservoir 50. Housing 49 has an internal diameter of about 9.7 mm along its length that is traveled by piston 52. The distal, outlet end of cartridge reservoir 50 is sealed by a septum 54 held by a cap 56 that is secured to a stepped-down diameter neck portion of the cartridge housing. When pen-needle assembly 38 is mounted on the distal end of a cartridge retainer 28 holding cartridge 48, the proximal point of injection needle 40 penetrates cartridge septum 54 to provide a fluid flow outlet by which medicine within cartridge reservoir 50 can be dispensed from the needle during operations of injection pen 20.

The fluid medicine container shown and described above is illustrative and not intended to be limiting as other constructions known in the art may be employed within the scope of the invention. For example, rather than having a distinct, disposable cartridge held within a separate, reusable retainer as in the shown fluid container, a fluid container may be provided in the form of a disposable cartridge constructed to be sufficiently durable and adapted to secure directly to pen proximal portion 24 without any protective retainer therearound, and with a pen-needle assembly directly mountable to that cartridge.

Pen proximal portion 24 includes an external protective housing 60 having an exterior on which are accessible display 62, such as a liquid crystal display, and input buttons 64, 66, and 68. Input buttons 64 and 66 may be pressed by a user to increment and decrement, respectively, a counter within an electronic circuit of pen 20 to adjust the dose of medication to be injected by pen operation. Input button 68 may be pressed by a user to, for example, scroll through data shown on display 62, such as data stored in pen memory related to times and dates of one or more previous doses, and the like. Actuator 70 at the proximal end of proximal portion 24 is designed to be plungeable relative to housing 60 to send a signal to the electronic circuit to cause the medicine to be dispensed as described below.

FIG. 2 shows components of pen 20 including a motorized driver assembly indicated generally at 90 that is mounted within housing 60 and which is used to advance a drive member into the cartridge 48 so as to cause medicine to be outlet therefrom. The drive member is in the form of a plunger 75, made in one piece from plastic or metal, having a distal, solid end face 77 that during use abuts and thereby directly engages piston 52 of cartridge 48. Rearward of end face 77, plunger 75 has a cylindrical tubular body 79. Along its entire axial length, tubular body 79 defines an internal hollow or blind bore 80, which is also cylindrical in the shown embodiment. The exterior of tubular body 79, as well as internal hollow 80, may be shaped other than cylindrical within the scope of the invention. Along the internal surface of tubular body 79, a helical thread 82 is formed. Plunger body 79 is sized to freely insert within the interior of cartridge 48, and with sufficient length to move piston 52 distally to suitably dispense the medication from cartridge 48.

Drive member 75 is advanceable distally in the axial direction by the motorized driver assembly 90 that is coaxially aligned with the cartridge 48. In the shown embodiment, driver assembly 90 includes an electric motor 92, a gear train 94, and an externally threaded nut 98 that is connected to the gear train 94 via a thrust bearing or bushing indicated at 96 which absorbs the axial loading. The threading of nut 98 engages plunger threading 82. Along its entire length, driver assembly 90 has a transverse cross-sectional shape that is dimensioned so as to be fittable within the space occupied by the medicine cartridge 48.

Different types of electric motors may be used as part of the motorized driver assembly, including DC permanent magnet motors, DC brushless motors, and stepper motors. Gear train 94, such as a planetary gear train, is adapted to reduce a high-speed motor shaft output into a suitable rotation of the driver nut 98. In an alternate embodiment, gear train 94 could be eliminated if a suitable threading of the driver nut 98 and plunger were provided. Driver assemblies and cooperating drive members different from the one shown that uses a nut and an internally threaded plunger, such as a driver assembly configured to ratchet a drive member forward during operation, also may be used within the scope of the invention.

As apparent in FIG. 2, in which plunger 75 is shown fully proximally retracted prior to its first use to expel medication from cartridge 48, the length of motorized driver assembly 90 is nearly entirely housed within internal hollow 80. Internal hollow 80 need not be air or fluid tight as plunger body 79 may be apertured without compromising its function. For example, plunger body 79 includes a longitudinally extending slot 100 extending radially therethrough. Slot 100 serves as a keyway for an anti-rotation key described further below. Slot 100 need not extend through the body thickness to function as a keyway, and instead could be provided as, for example, a continuous groove.

By accommodating all of the axial length of each of nut 98, thrust bearing 96, and gear train 94, and a substantial portion of the axial length of motor 92, within the internal diameter of plunger 75, space within pen 20 is used efficiently. The portion of motor 92 shown extending proximally beyond the proximal end of plunger 75 may be larger in diameter, and further is directly mounted to housing 60, or alternatively to a not shown member that itself is fixedly connected to the housing, for purposes of insuring that motor 92 does not rotate relative to housing 60 during pen operation. As motor 92 is rotatably and axially fixed to the housing, and plunger 75 is also rotatably fixed to the housing as described below, rotation of nut 98 caused by motor operation produces an axial motion of plunger 75 relative to the housing. In alternate embodiments, lesser length portions of the motorized driver assembly may be disposed within hollow 80 when plunger 75 is fully retracted, but such would ordinarily require lengthening pen 20.

Plunger 75 is prevented from rotating relative to housing 60 by means of its keying thereto, such as via a keyed slider generally indicated at 115, or alternatively by a key integrally formed with the housing. The shown slider 115 is in the form of a spring-loaded annulus 117 having a radially inwardly projecting key 119 that slides within the longitudinally extending slot 100 in plunger body 79. Annulus 117 is in turn keyed to the housing 60 with one or more not shown keys to be rotatably fixed and axially shiftable therein. The distal face of slider annulus 117 abuts cartridge 48 and urges that cartridge forward or distally when the fluid container is mounted to the pen proximal portion 24. Annulus 117 is biased forward by a metal, coiled compression spring 120 that has one end abutting slider 115, and the opposite end abutting a shoulder 61 of housing 60.

Operation of electric motor 92 is controlled by an electronic circuit, including a micro-controller, mounted within housing 60 and abstractly represented at 105. As represented in FIG. 2, electronic circuit 105 is electrically circuited with input buttons 64, 66 and 68 to allow the user to input information to or access directly the electronic circuit 105 for controlling operation of pen 20. Electronic circuit 105 is also electrically circuited with display 62 and actuator 70, and all the electrical components are powered by one or more batteries represented at 110 and mounted within protective housing 60.

Although actuator 70 is shown as being an axially projecting, plungeable and non-rotatable knob, other forms of actuators may be employed within the scope of the invention. For example, the actuator may comprise a rotatable knob otherwise similar to actuator knob 70, but which is twistable to set a dose for delivery rather than using buttons 64 and 66. Still further, the actuator may be a thumb wheel projecting beyond the proximal end of pen 20. The thumb wheel may be rotated to increase or decrease the dose to be delivered rather than using dedicated buttons 64 and 66, and that thumb wheel may be mounted in housing 60 to be axially plungeable as a unit to start the injection.

To operate pen 20, a user manipulates input button 64 and possibly input button 66 to signal to electronic circuit 105 the dose desired to be delivered, which dose is displayed on display 62. For priming the pen prior to use, a small dose is typically set by the user and then actuator 70 is manually plunged by a user while the user points the needled pen end upward, which actuator plunging causes the pen to internally operate in the manner described further below with respect to an actual injection. After priming, a user then sets the dose desired to be delivered with the input buttons, moves pen 20 such that needle 40 is brought into contact with the desired injection site of the user, and presses actuator 70. The plunging of actuator 70 causes an electric signal to be received by electronic circuit 105, and the circuit 105 causes motor 92 to operate. The motor output causes a rotation of nut 98, which due to its threaded engagement with the rotatably fixed plunger results in an advancement of plunger 75 in the distal direction that shifts piston 52 to deliver the selected amount of medication. If pen 20 were adapted to deliver a single fixed dose, the inputs of the dose selection could be eliminated, or reduced to merely the selection of either a priming dose or the fixed dose, and the pen could cause an appropriate dose to be delivered upon each plunging or other triggering of the actuator.

Electronic circuit 105 is able to control the position of plunger 75 based on the control by the circuit of the operation of motor 92. In particular, one suitable motor includes three evenly angularly spaced Hall-effect sensors that produce three signals per each motor shaft revolution, which signals are transmitted to the electronic circuit 105. Electronic circuit 105 uses these motor signals to control plunger advancement during a given injection operation, as well as possibly to sum up how far the plunger has been advanced since a new cartridge was installed to track medicine remaining. Such a motor is available as Maxon EC6-215550 from Maxon Motor AG located in Sachseln, Switzerland, and has a diameter of approximately 6 mm and a length of approximately 21 mm. A suitable gear train for use with this motor is known as Maxon GP6-199690, also available from Maxon Motor AG. The overall length of the above-identified Maxon motor with this Maxon gear train attached is approximately 35.9 mm.

Other suitable motors that work with an appropriate electronic circuit may be employed, such as a motor having an optical encoder that senses motor rotation, or a stepper motor with either open or closed-loop control.

Plunger 75 is preferably provided with a not shown microswitch that, when the plunger has been fully extended to empty cartridge 48, causes a signal to be sent to electronic circuit 105. This signal prompts electronic circuit 105 to operate motor 90 in reverse to fully retract plunger 75, as well as to cause a message or icon to be shown in display 62 as to the need for a replacement cartridge. The user can then remove the fluid container, replace the spent cartridge with a new cartridge, and then reassemble the pen for subsequent operation. In an alternate pen, a microswitch could instead sense when the cartridge has been removed and then retract the plunger automatically.

The pen also could be configured so as to monitor motor current/speed to automatically sense when the plunger 75 contacts piston 52. This sensing would occur automatically when the cartridge is replaced and minimizes the user's efforts needed to prepare the pen for its initial use with the new cartridge.

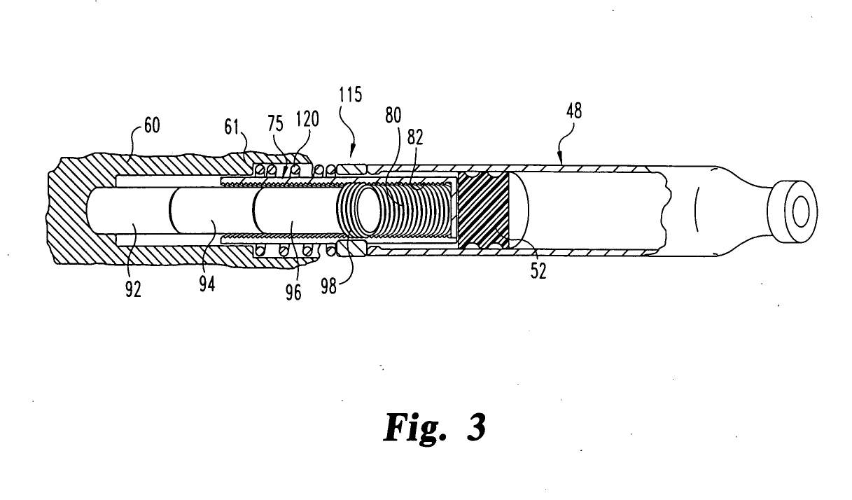

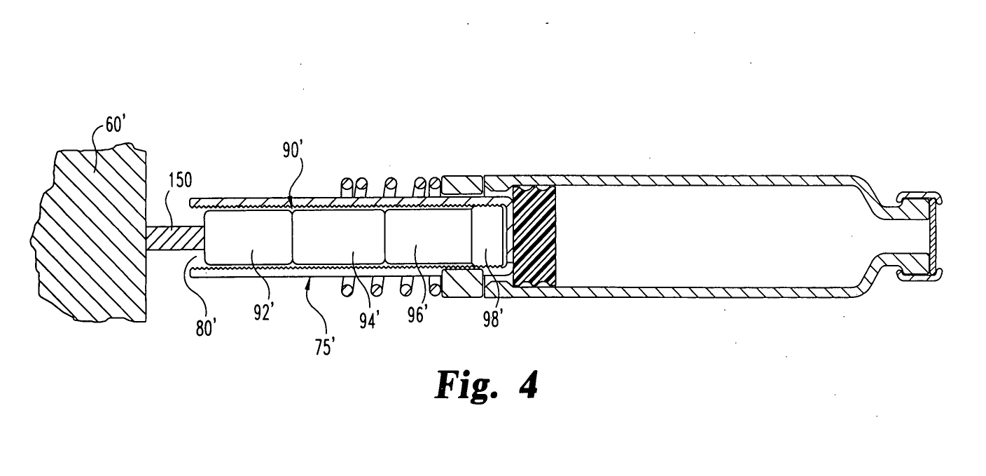

Referring now to FIG. 4, there is shown an alternate embodiment of the present invention, wherein certain parts similar to those shown in the embodiments of FIGS. 1-3 are labeled with a prime reference. This embodiment is identical to that of FIG. 2, except that the motorized driver assembly 90′ is further reduced in length so as to completely fit within the internal hollow 80′ of plunger 75′ when the plunger is fully retracted as shown in FIG. 4. In order to axially and rotatably retain the motorized driver assembly 90′, a support 150 axially extends into hollow 80′ of plunger 75′ to fixedly connect the motor 92′ to the housing abstractly indicated at 60′. Otherwise formed supports may naturally be provided within the scope of the invention.

While this invention has been shown and described as having multiple designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

1. A medication injecting apparatus comprising:
a housing;
a drive member movable in a distal direction relative to said housing from a retracted position to an extended position;
a fluid container defining a medicine-filled reservoir with a movable piston at one end and an outlet at the other end, said piston engagable by said drive member to be shifted distally toward said outlet when said drive member is moved distally;
an electronic circuit;
a motorized driver assembly controlled by said electronic circuit and coupled with said drive member for selectively shifting said drive member distally; and
characterized in that said drive member includes a tubular body that inserts within said fluid container when advancing from said retracted position to said extended position, said tubular body defining an internal hollow; and
wherein at least a portion of said motorized driver assembly fits within said internal hollow when said drive member is disposed in said retracted position.

2. The medication injecting apparatus of claim 1 wherein said motorized driver assembly comprises an electric motor, and wherein at least a portion of said electric motor fits within said internal hollow when said drive member is disposed in said retracted position.

3. The medication injecting apparatus of claim 2 wherein at least a majority of said electric motor fits within said internal hollow when said drive member is disposed in said retracted position.

4. The medication injecting apparatus of claim 3 wherein all of said electric motor fits within said internal hollow when said drive member is disposed in said retracted position, and wherein said electric motor is operatively attached with said housing by a support member that extends into said internal hollow when said drive member is disposed in said retracted position.

5. The medication injecting apparatus of claim 3 wherein said electric motor, along its entire length extending in the distal direction, has a transverse cross-sectional shape dimensioned so as to be fittable within the space occupied by said fluid container.

6. The medication injecting apparatus of claim 1 wherein a surface of said body defining said internal hollow is threaded, and wherein said motorized driver assembly comprises an externally threaded nut in threaded engagement with said threaded surface.

7. The medication injecting apparatus of claim 6 wherein said motorized driver assembly further comprises an electric motor, said nut located distally of said electric motor, and wherein at least a portion of said electric motor fits within said internal hollow when said drive member is disposed in said retracted position.

8. The medication injecting apparatus of claim 7 wherein said motorized driver assembly further comprises a gear train disposed between said electric motor and said nut, and wherein said gear train fits within said internal hollow when said drive member is disposed in said retracted position.

9. The medication injecting apparatus of claim 6 wherein said drive member and an electric motor of said motorized driver assembly are rotatably fixed relative to said housing.

10. The medication injecting apparatus of claim 6 wherein said drive member comprises an axially extending keyway in an external surface, and wherein a key that slides within said keyway while preventing rotation of said drive member is one of integrally formed with and rotatably fixed to said housing.

11. The medication injecting apparatus of claim 10 wherein said key radially inwardly projects from a rotatably fixed, spring-biased slider that urges said fluid container distally relative to said housing to which said fluid container is removably mounted.

12. The medication injecting apparatus of claim 1 further comprising a manually operable actuator electrically circuited with said electronic circuit, whereby responsive to an electrical signal received when said actuator is operated, said electronic circuit activates said motorized driver assembly to shift said drive member distally.

13. The medication injecting apparatus of claim 12 further comprising means for a user to input to said electronic circuit a dose to be delivered by operation of said apparatus, whereby said electronic circuit controls the shifting of said drive member based upon the input.

14. The medication injecting apparatus of claim 12 wherein said actuator comprises a plungeable element disposed at a proximal end of said housing.

15. A medication injecting apparatus comprising:
a housing;
a drive member movable in a distal direction relative to said housing from a retracted position to an extended position;
a replaceable cartridge defining a medicine-filled reservoir with a movable piston at one end and an outlet at the other end, said piston engagable by said drive member to be shifted distally toward said outlet when said drive member is moved distally;
an electronic circuit;
a motorized driver assembly including an electric motor, said motorized driver assembly controlled by said electronic circuit and coupled with said drive member for selectively shifting said drive member distally; and
characterized in that along its length extending in the distal direction, said motorized driver assembly is aligned generally coaxially with said cartridge; and
along at least a substantial portion of its length, said motorized driver assembly has a transverse cross-sectional shape dimensioned so as to be fittable within the space occupied by said cartridge.

16. The medication injecting apparatus of claim 14 wherein said at least a substantial portion of its length comprises the entire length of said motorized driver assembly.
